(19) United States
(12) Patent Application Publication
(10) Pub. No.: US 2005/0148088 A1
(43) Pub. Date: Jul. 7, 2005

(54) Pressurized hot water extraction

(76) Inventor: Eng Shi Ong, Singapore (SG)

Correspondence Address: Law Offices of Albert Wai Kit Chan, World Plaza Suite 604, 141 07 20th Avenue, Whitestone, NY, 11357, US

(21) Appl. No.: 10/511,925

(22) PCT Filed: Apr. 19, 2002
(86) PCT No.: PCT/SG02/00063

(51) Int. Cl. G01N 33/00

(52) U.S. Cl. 436/96

(57) ABSTRACT

Methods for the pressurized hot water extraction (PHWE) of compounds from a sample of interest are disclosed. Applications of the method to the extraction of bioactive compounds from botanical samples are disclosed. The disclosed methods do not require the use of subcritical conditions and may be conducted under dynamic flow conditions in the presence of one or more organic solvents. The disclosed methods also provide extraction efficiencies comparable to soxhlet extraction.

### TECHNICAL FIELD

The present invention relates to methods for extracting chemical compounds from a sample material by the use of pressurized hot water as a solvent at temperatures below 100 degrees Celsius. The invention utilizes less energy than “subcritical water” extraction methods and may be used to extract bioactive components from materials such as, but not limited to, botanical samples, and herbal preparations. The pressurized hot water may be used under dynamic flow conditions and/or supplemented with an organic solvent. The present invention is referred herein as “pressurized hot water extraction” or PHWE.

### BACKGROUND ART

Botanical drugs, classes of nutraceuticals and herbal preparations are medicinal products containing of a single plant or a mixture of 2 or more different types of medicinal plants. Monographs of medicinal plants can be found in the United States Pharmacopeia [1], Chinese Pharmacopeia [2], WHO monographs for medicinal plants [3], Japanese Pharmacopeia [4], Herbal Medicine (expanded Commission E monographs) [5] and others. For botanical drugs and herbal preparations, there is a need to approach scientific proof and clinical validation with chemical standardization, biological assays, animal models and clinical trials. Quality assurance of botanical drugs and herbal preparations is the prerequisite of credible clinical trials. According to draft guidelines by the United States Food and Drug Administration (U.S. FDA) [6] and The European Agency for the Evaluation of Medicinal Products [7], various aspects of analysis must be performed for the purpose of certification of botanical drugs and herbal preparations and chemical standardization is an important aspect.

For the chemical standardization of botanicals and herbal preparations, the extraction of bioactive components or marker compounds is an important step. The methods found in the monographs of Pharmacopeias and other reports [8,9] often used extraction methods that required significant volume of organic solvent and were rather tedious. Hence, methods that are rapid, required low volume of organic solvent and with high extraction efficiency are attractive options.

One of the problems for the chemical standardization of medicinal plants was that most of the target analytes were reasonably polar and thermally labile. From our earlier work and present studies [10], some of the target analytes such as berberine chloride and glycyxrhizin showed signs of degradation in the temperature range of 120 to 160° C. Additionally, they were present naturally where significant analyte-matrix interactions were present. Spiking of target analytes into the plant matrix does not mimic the analyte-matrix interaction present naturally. A high recovery obtained in the spiking experiments thus does not necessarily indicate the same results with a non-spiked sample.

In the interest of reducing the usage of organic solvent in analytical methods, sub-critical water extraction had been developed for the extraction of organic pollutants from environmental solids [11, 12, 13]. At the same time, an approach using static—dynamic sub-critical water extraction of essential oil components from plant materials was developed [14,15]. Superheated water under pressure between 125 to 175° C. had been used to rapidly extract oxygenated fragrance and favor compounds from rosemary [16]. A laboratory made system using pressurized hot water was developed for the extraction of iridoid glycosides in plant matrix with final determination by micellar electrokinetic capillary chromatography. The authors concluded that pressurized hot water extraction was not as efficient as hot water extraction for catalpol and aucubin in plant materials [17]. Subcritical water between 100 to 175° C. with an applied pressure of 50 bar were used for the extraction lactones from a kava root with final determination by gas chromatography [18]. These approaches utilized subcritical water under high temperature, high pressure, and/or static conditions for the extraction of semi-volatile components from plant materials. Other approaches also using high temperature, high pressure, and/or static conditions have also been described [25].

Citation of the above documents is not intended as an admission that any of the foregoing is pertinent prior art. All statements as to the date or representation as to the contents of these documents is based on the information available to the applicant and does not constitute any admission as to the correctness of the dates or contents of these documents.

### DISCLOSURE OF THE INVENTION

The present invention provides a simple system using pressurized hot water for the extraction of thermally labile and polar components from a sample. The methods of the present invention are referred to as pressurized hot water extraction, or PHWE, and are preferably applied to material from medicinal plants. The present invention provides an efficient and advantageous means to extract compounds of interest 1) in the absence of large quantities of organic solvents, 2) without excessive energy to heat water above 100° C., and 3) without exposing temperature sensitive compounds to excessive temperatures.

The present invention provides methods for using pressurized and heated water to extract one or more than one analyte of interest from a sample under pressures below about 30 bar and at temperatures below 100° C. The methods include, but is not limited to, use of PHWE to extract a solid or semi-solid analyte containing sample in an extraction cell under dynamic conditions. The water solvent is preferably subject to a heating step prior to contact with the analyte containing sample to maintain the temperature and pressure of the invention. The sample and/or extraction cell is preferably also -heated. When practiced in a dynamic mode, the extracted analyte is constantly extracted and removed by the continuing flow of heated and pressurized water moving through the sample and to a collection means.

In one aspect of the invention, PHWE is conducted dynamically at a flow of, or about, 1 ml/minute, a temperature below 100° C., an applied pressure ranging from above 1 to 30 bar, and an extraction time of about 40 minutes. In preferred embodiments, the temperature is at or about 95° C. while the pressure ranges from about 10 to 25 bar. In other embodiments of the invention, the temperature is preferably above 25° C. while the pressure is regulated by a regulator or constrictor on the flow of the heated water as a solvent Because the water temperature remains below 100° C., the applied pressure is not necessary to maintain the water in a liquid state. Instead, it results in the water being a compressed liquid during the extraction process.

In another aspect of the invention, the heated water used as the solvent may be supplemented by the addition of organic modifiers such as, but not limited to, ethanol to improve the extraction process. The amount of organic modifiers may range from 0 to about 50% (v/v), although preferred embodiments of the invention utilize heated and pressurized water with 0, 5, 10, 15, 20, 25, 30, 35, or 40% organic modifier.

Alternatively, the heated water may be supplemented by the addition of one or more surfactant or detergent to improve the extraction process. Surfactants are soluble agents that reduce the surface tension of liquids and/or reduces interfacial tension between a liquid and a solid or between two liquids. Detergents have the ability to emulsify solids and liquids to suspend them in solution in a liquid. The present invention may be practiced with anionic, cationic, zwitterionic (amphoteric) and nonionic (polar) surfactants and detergents and combinations thereof Examples of surfactant or detergent for use in the practice of the invention (alone or in combination) include, but are not limited to, sodium dodecyl sulfate (SDS); caprylic acid, sodium salt; cholic acid, sodium salt; 1-decanesulfonic acid, sodium salt; deoxycholic acid, sodium salt; glycocholic acid, sodium salt; glycodeoxycholic acid, sodium salt; taurocholic acid, sodium salt-taurodeoxycholic acid, sodium salt; cetylpyridinum chloride; dodecyltrimethylammonium bromide; hexadecyltrimethylammonium bromide; tetradecyltrimethylammonium bormide; CHAPS; CHAPSO; n-decyl β-D-glucopyranoside; digitonin; n-dodecyl β-D-glucopyranoside; n-dodecyl β-D-maltoside; n-heptyl β-D-glucopyranoside; n-octyl β-D-glucopyranoside; n-octyl α-D-glucopyranoside; Nonidet P40; n-nonyl β-D-glucopyranoside; and Triton X-100. The amount of surfactant or detergent may range from 0 to about 10 or about 20 or about 30 or about 40 or about 50 or about 60 or about 70 or about 80 or about 90 or about 100 mM. Alternatively, the amount of surfactant or detergent may be less than about 1% (v/v or w/v), preferably less than about 0.75% or less than about 0.5% or less than about 0.25% or less than about 0.1% or less than about 0.05% or less than about 0.01%. The amount of surfactant or detergent to use in the practice of the invention may be determined by the skilled person by routine experimentation.

While the present invention is exemplified by the extraction of marker compounds such as berberine from coptidis rhizoma, glycyrrhizin from radix glycyrrhizae/liquorice and baicalein from scutellariae radix (FIG. 1)

In addition to the extraction of compounds from coptidis rhizoma, radix glycrrhizae/liquorice and scutellariae radix, the present invention maybe applied to the extraction of compounds from any plant or herbal material and combinations thereof. Non-limiting examples of plant material include rosemary, oregano, lemons, oranges, mushrooms, rice, fennel, cinnamon, sage, lavender, marjoram leaves, kava root, laurel leaves, calendula, valeriana, curcuma, and pulps thereof. Especially preferred in the practice of the present invention are plant materials contain compounds with medicinal or nutritional value.

Also preferred in the practice of the invention are plant materials containing compounds such as, but not limited to, essential oils and/or fragrances. Non-limiting examples of such compounds include, but are not limited to, β-pinene, β-myrcene, α-phelandrene, camphor, terpinen4-ol, linalyl propanoate, anethol, copaene, caryophyllene, eugenyl acetate, d-limonene, carvone, eugenol, 1,8-cineole, nerol, and α-pinene.

The extraction efficiencies of the invention's methods compare favorably with that using sonication and soxhlet extraction for different medicinal plants. PHWE gives extraction efficiencies comparable to soxhlet extraction for baicalein from scutellariae radix, to sonication for berberine from coptidis rhizoma, and to sonication for glycyrrhizin from radix glycyrrhizae. Pressurized liquid extraction (PLE) using methanol was also used for extraction of baicalein from scutellariae radix in comparison to PHWE.

The present-invention may also be applied to the extraction of pesticides, herbicides, pollutants and other toxic compounds that may be present in plant material. Such an application is particularly advantageous for extraction and detection/identification of such compounds in plant materials.

Samples for extraction by the present methods are preferably combined with a dispersant to prevent, minimize or reduce sample compaction due to pressure and water absorption. Compaction may result in impediments and/or interruptions (e.g. clogging) in the flow of solvent water during extraction. The inclusion of a dispersant may also provide more contact between solvent water and a sample during extraction by reducing the amount of sample-sample contacts. Preferred dispersants for use in the practice of the present invention include, but are not limited to, sand, glass beads, diatomaceous earth, and Celite.

The methods of the present invention may be practiced with an apparatus as described in U.S. patent application Ser. No. 09/991,151, filed Nov. 16, 2001, which is hereby incorporated by reference as if fully set forth In preferred embodiments of the invention, the present invention is practiced by use of an extraction apparatus coupled with means to conduct analytical methods such as capillary electrophoresis (CE), including capillary zone electrophoresis (CZE), high performance liquid chromatography (HPLC), gas chromatography, mass spectroscopy and others to provided an efficient, accurate and environment-friendly means for the chemical standardization of analytes from a sample. The availability of these analytical methods permits compounds present in sample extracts to be quantitatively and qualitatively determined. This is particularly advantageous and beneficial for the extraction of compounds from medicinal plant materials.

### BRIEF DESCRIPTION OF THE DRAWINGS

FIG. 1, parts A, B and C show the chemical -structures of glycyrrhizin, berberine and baicalein, respectively.

FIG. 2 shows the effect of temperature on extraction efficiencies of baicalein from medicinal plants by PLE part A) and the effect of added ethanol on the extraction efficiencies of baicalein from medicinal plants by PHWE (part B).

FIG. 3 shows the effect of added ethanol on the extraction efficiencies of glycyrrhizin from medicinal plants by PHWE.

FIG. 4 shows the effect of temperature (part A) and ethanol (part B) addition on the extraction efficiencies of berberine from medicinal plants by PHWE.

FIG. 5 shows chromatograms obtained for extraction of baicalein from scutellariae radix by PLE (part A), baicalein from scutellariae radix by PHWE (part B), glycyrrhizin from radix glycyrrhizae/liquorice by PHWE (part C), and berberine from coptidis rhizoma by PHWE (part D).

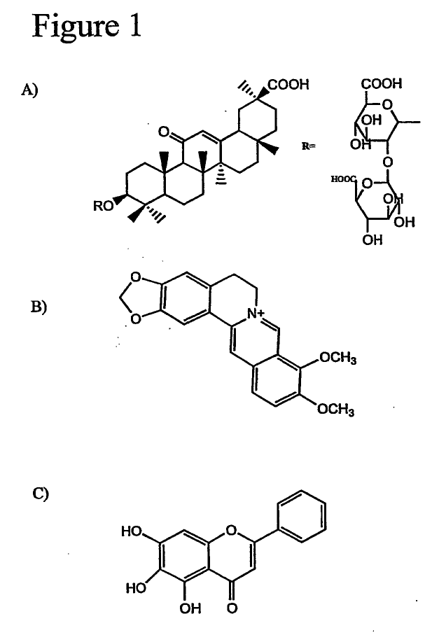

### MODES FOR CARRYING OUT THE INVENTION

The present invention provides pressurized hot water extraction methods for extracting one or more than one organic compounds from a sample of interest by use of a system applying heated water under pressure. The invention may be used to extract a diverse array of organic compounds to be used as analytes for further analysis by means such as capillary electrophoresis (CE) or high performance liquid chromatography (HPLC). The invention may thus be utilized in, or as part of, an analytical standardization protocol for determining the presence of one or more compounds in the sample.

The method (and apparatus) of the invention may also be used as a preparative means to extract one or more compounds of interest from a sample. Whether used in an analytical or preparative mode, the invention provides one or more than one extracted compound which may be further purified to provide a means for the manufacturing of a compound or compounds for further analysis, use in trials and assays, formulation of pharmaceuticals or other consumable health supplements, and other uses recognized by one skilled in the art. In one embodiment of the invention, the methods provided an efficiency and accurate means for the chemical standardization of analytes from a sample of interest.

Extraction of active ingredients is an important step which can affect the accuracy of the method in the determination of analytes present naturally in medicinal plant material. In most reports, the extraction steps are often long and tedious with high solvent consumption. PHWE as disclosed herein is an advantageous method compared to traditional methods of extraction such as soxhlet and ultrasonic for the extraction of target analyte in botanical samples. PHWE uses only water, or water and a small proportion of an organic modifier at elevated temperatures with applied pressure to extract compounds from a sample. The applied pressure helps to force the solvent into the pores of the sample matrix but is not necessary to keep the solvent water in a liquid state because the temperature is below 100° C. Extraction with the present invention also appears to disrupt analyte-matrix interactions and provides advantages in time and energy savings as well as lower solvent consumption.

One aspect of the present invention addresses the fact that the determination of active ingredients in medicinal plants is complicated by the lack of certified reference materials. The accuracy of any method is difficult to determine as spiking of the target analyte into the medicinal plant will not mimic the analyte-matrix interaction present naturally. Furthermore, the high recoveries observed in spilling experiments would not imply that the method was accurate. Additionally, determination of active ingredients in herbal preparations/CPM are more difficult as they are known to contain between 2 to 10 different types of medicinal plants. In one embodiment of the invention, a single step extraction using PHWE with no further cleanup is provided.

In another embodiment, the present invention may be used to extract berberine. Berberine is a common alkaloid found in medicinal plants such as rhizoma coptidis (huang lian) and species of mahonia It has been reported to counteract toxicity, exhibit antibacterial and antiinflammatory activity. Due to berberine's therapeutic value, the determination of its levels in medicinal plants, Chinese prepared medicine (CPM) and health supplements are of importance.

The Chinese pharmacopeia includes a method for the analysis of berberine in medicinal plants using extraction with methanol on a water bath at 60° C., sonication for 30 minute and allowed to stand overnight with final analysis by TLC. Multiple step ultrasonic extraction has also been used to extract berberine in medicinal plants and related herbal preparations with analysis by HPLC and HPLC/MS respectively. Similarly, berberine in medicinal plants or CPM products are determined using ultrasonic extraction with CZE and CE-ESI-MS.

A “sample” as used herein is a solid or semi-solid material containing the compounds of interest and to be extracted. As noted above, one or more than one of the compounds of interest may be an analyte of interest, such as, but not limited to, by being a pharmaceutically active compound; a toxic substance, a contaminant or impurity in the source material sampled; or an additive to the sample. The compound may optionally be the major or main component of the sample. For example, non-limiting compounds from medicinal plants; herbal preparations; food products; aquatic samples including fish or shellfish; waste materials, sediments or sludges; soils; or animal and plant tissues such as leaves, cellulose products, roots, and bark may all be extracted by use of the present invention. Contaminants or impurities in samples of medicinal products, foods, and industrial reagents may also be extracted. Concentrations, in absolute or relative terms, of the main or major components of various samples may also be extracted.

An extracted compound/analyte of the invention is preferably organic, or generally more soluble in organic or non-aqueous solvents than in water or other aqueous solvents. They are also preferably bioactive. The compounds/analytes include, but are not limited to, berberine, aristolochic acids, strychnine, ginsenosides, glycyrrhizin, baicalein, other compounds of the Chinese Phamacopoeia or other Pharmacopoeia monographs, food additives, vitamins, other pharmaceutical compounds, drugs, hormones, lipids, (organophosphorus) pesticides, herbicides, polychlorinated biphenyls (PCBs), polycyclic aromatic hydrocarbons (PAHs), gasoline components, triglycerides, phenols, aldehydes, alcohols, lipids, waxes, nitrosamines, phthalates, halogenated esters, hydrocarbons, chlorinated hydrocarbons, heterocyclic compounds, phosphates, acids, bases, polymer additives, or mixtures thereof.

In one embodiment of the invention, the samples contain solid particles obtained from a source material of interest by cutting, scraping, crushing, grinding, pulverizing and/or other sampling means known in the arts Samples may also be extracted after dehydration treatment, although a drying step is not necessary for inclusion in the present invention. If a drying step, including but not limited to heat treatment, evaporation, or treatment with desiccants such as acetone or ethanol, is included the destruction or loss of volatile analytes should be minimized.

Samples that are composed of fine particulates are more likely to improve penetration and channeling of the solvent through the sample to result in better extraction. Alternatively, a dispersant may be combined with the sample to improve the extraction. process. During preparation of the sample, excess heating should be minimized to prevent loss of volatile analytes or otherwise changing the amounts or chemical nature of the analytes. The samples mayalso optionally be spiked with one or more than one known compound, preferably in known quantities or concentrations, as an internal reference standard for the extraction process or the extraction profile.

As noted herein, water is the solvent used for PHWE, and an optional organic modifier may be included in the water to improve extraction. A diverse range of organic modifiers and combinations thereof may be utilized in the present invention. The choice of organic modifier depends on the analyte to be extracted and the nature of the sample as well as the skill and experience of the practitioner, although modifiers that are soluble in water are of course preferred.

Suitable modifiers include, but are not limited to, C₁-C₆ alcohols, ketones, ethers, alcohol ethers, amides, sulfoxides, carbonates, aldehydes, carboxylic acids, nitrites, and acetamides. The amount of organic modifiers may range from 0 to about 50% (v/v), although preferred embodiments of the invention utilize heated and pressurized water with 0, about 5, about 10, about 15, about 20, about 25, about 30, about 35, or about 40% organic modifier.

For a sample containing one or more unknown analytes, the choice of a suitable modifier may be made in a variety of ways. The sample may be divided up and extracted using different types and concentrations of modifiers. A determination of the best modifier for a particular analyte may be made by comparison to known extraction profiles. Alternatively, a sample may be repeatedly extracted using different types and concentrations of modifiers, and the extracted analytes compared. This is usually performed sequentially using a series of solvents with a uni-directional change in a particular characteristic. An example is the sequential use of a non-polar, a slightly polar, and a highly polar modifier.

Briefly, extraction with the present invention may be summarized as follows. A pump is operably linked to an extraction cell, comprising a sample compartment which may be contacted by solvent (water) moving through the cell, such that the pump may be used to deliver the solvent to said extraction cell containing the sample to be extracted. Said extraction cell comprises an input connector which is attached to the solvent line leading from the pump to the extraction cell. The solvent line leading from the pump is contacted with a preheating element, such as a heating coil, or otherwise heated to bring the solvent to the desired temperature prior to contact with the sample. The heated solvent proceeds through the extraction cell and leaves it through an output connector which is attached to a solvent line leading away from the extraction cell. The heated solvent proceeds under a dynamic mode through a backpressure regulator, which is operably linked to the extraction cell via said solvent line and output connector, and then to a collection means. The backpressure regulator is thus capable of regulating the pressure of the solvent in the extraction cell to keep the solvent in a liquid state as it dynamically contacts the sample in the extraction cell. As used herein, “operably linked” refers to a physical arrangement between components that permit them to function in their intended ways in an PHWE apparatus of the invention.

In one preferred embodiment of the invention, the extraction cell and solvent line leading from the pump are maintained in a heating assembly capable of heating said extraction cell and said solvent line (and optionally a preheating element in contact with said solvent line). One non-limiting example of such a heating assembly is an oven.

As used herein, “regulate” or “regulated” refers to the directing and/or controlling of the solvent pressure in the PHWE method of the invention. Preferably, this pressure is “regulated” to be at a constant pressure or within a particular range of pressures. The pressure is thus not permitted to vary beyond or below a set point (or limit) or beyond or below a set range. While this may be accomplished by a variety of means, one non-limiting means is by directing, controlling and/or adjusting the amount, rate, and/or flow of solvent in the PHWE method of the invention.

The method of the invention proceeds generally as follows. First, the extraction cell is loaded with sample to be extracted. The sample contains the analyte or analytes of interest. In a preferred embodiment, the sample fills the cell, that is, the dead volume of the cell is 10% or less. A dispersant may be added to occlude any voids in the sample after placement in the cell. Compression or expansion of the sample during extraction may occur to change the dead volume. The size of the extraction cell is thus preferably selected to allow the sample to fill the cell completely. Suitable extraction cells have volumes of about 0.1 ml to about 50 ml, with about 0.5, about 1, about 2, about 5, about 10, and about 15 ml extraction cells being preferred. Other sizes may also be used. The extraction cells are of course composed of materials which allow the use of the solvents, pressures and temperatures of the invention. Suitable extraction usually have fruits of some type to retain the sample in the cell, as will be appreciated by those skilled in the art.

Alternatively, the sample does not fully fill the volume of the extraction cell, and an inert filler is used. In some cases, inert fillers may be mixed into the sample if it is highly compressible, which can lead to clogging of the system. Suitable inert fillers include solid (particulate) substances which do not contain extractable materials, such as sand, diatomaceous earth or glass wool. Other inert fillers are known by the skilled artisan Glass wool or other similar means may be used in combination with a dispersant and placed between the sample and the outlet for solvent water leading away from the sample as well as the inlet for solvent water to reach the sample.

Once the extraction cell is loaded with sample, it is attached via its inlet and outlet to be in the solvent line between the pump and the collection means. Preferably, the extraction cell is contacted with the solvent and placed within a preheated oven or heating block and allowed to equilibrate to the oven or block temperature. Alternatively, the extraction cell may be exposed to preheated solvent immediately after placement in the oven or heating block. Preheating of the solvent is necessary to generate the appropriate pressure in the system as outlined below. Extraction preferably proceeds in a dynamic, flow through mode.

Pressures used will depend on the particular solvents and samples of the run; for example, samples with high levels of extractable materials generally require less pressure. Suitably, the pressure ranges from about 10 bar to about 30 bar. Preferably, the range is from about 10 to about 20 or about 20 to about 30 bar. Particularly preferred pressures are at or about 10, at or about 15, at or about 20, or at or about 25 bar. The backpressure regulator may be set to prevent the pressure from exceeding a set upper limit. When used in combination with the temperature setting, the backpressure regulator may constrain the pressure in the system to be within a certain range and/or no more than an upper limit of interest. The adjustable backpressure regulator can also be used to reduce the pressure buildup in the system when it exceeds a set point.

The temperature used in the present invention is below 100 degrees Celsius, more preferably from about 30 to about 50, about 50 to about 60, about 60 to about 70, about 70 to about 80, about 80 to about 90, and about 90 to about 95 degrees Celsius. In particularly preferred embodiments of the invention, the temperature is at or about 95, at or about 94, at or about 93, at or about 92, at or about 91, at or about 90, at or about 85, at or about 80, at or about 75, at or about 70, at or about 65, or at or about 60 degrees Celsius. As with the pressure used, the exact temperature used will depend on the solvent(s) and the nature of the analyte(s) and sample.

The temperatures and pressures used in the method of the present invention are below “subcritical conditions” as commonly used in the art Stated differently, the solvent systems used are in liquid form even in the absence of the applied pressure. They are thus compressed liquids.

The extraction is conducted for a period of time with a constant flow rate of solvent(s) from the pump. Preferably, the time is sufficient for extraction of the analyte(s) of interest and/or about 40 minutes and the flow rate is at or about 1 ml/min. Alternatively, higher flow rates, ranging from about 0.1 to about 5 ml/min, such as but not limited to about 0.2, about 0.5, about 1.5, about 2, about 3, or about 4 ml/min may be used for times ranging from about 5, about 6, about 7, about 8, about 9, about 10, about 15, about 20, about 25, about 30, about 35, about 40, about 45, about 50, about 60, about 90, about 100, about 120, about 150, about 180 or about 200 minutes.

As well appreciated in the art, and generally observed, the faster the flow rate, the less efficient the extraction. Higher flow rates may be appropriate, however, for larger extraction cells or samples with large quantities of compound/analyte. Slower flow rates may be used to increase the degree of extraction or with samples containing small amounts of compound/analyte.

The time used for extraction may be chosen in several ways, and will depend in part on the purpose of the extraction. For example, a less efficient extraction, shorter time period, may be used if qualitative identification is the goal. A more complete extraction, longer time period may be used if quantitation or yield of the analytes is critical.

Preferably, the extraction is run such that not more than about 20%, and preferably not more than 10%, more of the analyte or analytes will be subsequently extracted in a subsequent extraction using the same method or other extraction methods such as accelerated solvent extraction, soxhlet or microwave extraction. The time of extraction is thus selected to extract about 80-90% of the extractable material from the sample. Generally, as outlined above, this time ranges from about 5 to about 200 minutes. One measure of adequate extraction is that no more than about a further 10% of the compounds/analytes are extracted by maintaining the same extraction conditions for an additional equivalent time period. As recognized by the killed artisan, sample extraction may be discontinuous, so the time factor is the total time of extraction

Preferably, the sample is not dissolved during extraction, but rather the analytes removed. The conditions of the reaction are thus designed to avoid the complete or substantial dissolution of a solid sample. However, solid samples containing significant amounts of extractable material may show a decrease in mass as a result of the extraction of the analytes.

Once the extracted compounds/analytes are collected, they may be subjected to further analysis. This may be done by any means known in the art and depending on whether identification or quantification of the analytes or both is of interest. The selection of analytical means also depends on the composition of the analytes. The compounds/analytes may be left in the solvent(s), or the solvent(s) removed, as part of the analysis. The analytes may be analyzed using techniques well known in the art, including, but not limited to, gas chromatography, mass spectrometry, ion chromatography, liquid chromatography or capillary electrophoresis. In addition, the solvent(s) containing the compounds/analytes may be concentrated prior to analysis, for example by inert gas blow-down or evaporation. If the concentration of compounds/analytes is high, they may also be diluted prior to analysis.

The following examples are put forth so as to provide those of ordinary skill in the art with a complete disclosure and description of how to make and use the present invention, and are not intended to limit the scope of what the inventors regard as their invention nor are they intended to represent that the experiments below are all and only experiments performed. Efforts have been made to ensure accuracy with respect to numbers used (e.g. amounts, temperature, etc.) but some experimental errors and deviations should be accounted for. Unless indicated otherwise, parts are parts by weight, temperature is in degrees Celsius, and pressure is in bar.

### EXAMPLE 1

### Materials and Methods

All reagents were of analytical grade. Berberine chloride, baicalein and glycyrrhizin were purchased from Sigma (St. Louis, Mo., USA). Methanol and ethanol was purchased from Hayman (Witham, Essex, England). Sand purified by acid (about 40 to 100 mesh) was purchased from BDH Chemical Ltd (Poole, England). Sodium dihydrogen phosphate and phosphoric acid were purchased from Merck (Darmstadt, Germany) and Hayashi (Osaka, Japan). Pure water was obtained from Millipore Alpha-Q water system (Mdlipore, Bedford, Mass., USA).

Stock solutions of berberine chloride, baicalein and glycyrrhizin at. 1000 mg/l were prepared in methanol respectively. For all analysis, the working solutions of berberine chloride, baicalein and glycyrrhizin were prepared in the range of 0 to 60 mg/l in methanol. Linearity of berberine chloride, baicalein and glycyrrhizin were established between respectively with correlation coefficient R≧0.99. For the quantitation of marker compounds, a three point calibration based on the linearity established were used.

To prepare a homogenous sample, the different types of medicinal plants were ground using an IKA MF10 microfine grinder (Staufen, Germany) with sieve insert of hole size 0.5 mm to produce sample powder. For all experiments, the sample powder was combined with purified sand as a dispersant. The extraction cells were finally filled with the sand to avoid any voids.

For pressurized liquid extraction as discussed below, 0.3 g of samples were weighed directly into the extraction cell as mentioned in PLE instrumentation. Methanol was used as the extraction solvent.

For pressurized hot water extraction (PHWE), 0.1 to 0.2 g of medicinal plant samples were weighed directly into a glass tube and mixed thoroughly with a high proportion of purified sand. The sand and plant material mixture was transferred into the extraction cell for extraction as described below. The extraction cells were finally filled with the sand to avoid any voids.

For glycyrrhizin, the procedures were adopted from an earlier report [10]. Briefly, 0.6 g of ground sample were extracted with 20 ml of methanol/water mixture (70:30) at room temperature for 10 minutes and centrifuge at 2000 rpm for 10 minutes. The procedures were repeated three times. The extracts were combined, excess solvent was evaporated with the rotary evaporator (Heidolph, Schwabach, Germany) and filtered through Whatman No 1 filter paper into a 50 ml volumetric flask.

For berberine, the procedures were adopted from an earlier report [24]. Briefly, 0.3 g of ground sample were extracted with 20 ml of methanol/water mixture (70:30) at room temperature for 10 minutes and centrifuge at 2000 rpm for 10 minutes. The procedures were repeated three times. The extracts were combined, excess solvent was evaporated with the rotary evaporator and filtered through Whatman No 1 filter paper into a 50 ml volumetric flask

### Soxhlet Extraction

For baicalein, 0.3 g of sample was weighed into the thimble. The solvent was selected based on the method mentioned in the Chinese Pharmacopeia [3]. The co-extract gave a yellow color with the extraction solvent The yellowish color turned lighter and lighter through the course of the extraction After extraction with 100 to 120 ml of methanol/water (70:30) for three to four hours, the extraction solvent was essentially colorless. The excess solvent was evaporated with the rotary evaporator and filtered through Whatman No 1 filter paper into a 50 ml volumetric flask.

Pressurized Liquid Extraction (PLE)/Pressurized Hot Water Extraction (PHWE System

The instrumentation used for pressurized hot water extraction is described in U.S. patent application Ser. No. 09/991,151, filed Nov. 16, 2001 and was also used for pressurized liquid extraction (see also references [19,20] below). Briefly, the stainless steel tubings used were 1/16 inch OD and 0.18 mm ID. The back pressure was generated using a back pressure regulator by VICI Jour Research (Onsala, Sweden). The extraction cells were of stainless steel with 10 mm ID×150 m (approximately 10 ml). The extraction cell was heated in a HP5890, gas chromatograph oven (Hewlett Packard, USA). The pump used was a ternary gradient HP1050 HPLC pump Hewlett Packard, Waldbronn, Germany). The pump flow was set at 1.0 ml/min and the oven temperature was set at 100° C. or below. The pressure in the system indicated by the HPLC pump was between 10 to 30 bar. The extraction cell was prefilled with water to check for possible leakage before setting the temperature of the oven to the required value. Extraction with, water was carried out for a period of 40 minutes and 40 to 45 ml of (analyte containing) solvent was collected. In between runs, the system was washed with water for 5 minutes. The excess liquid collected was evaporated with the rotary evaporator and filtered through Whatman No 1 filter paper into a 25 or 50 ml volumetric flask.

For baicalein from Scutellariae, methanol was used as the solvent for pressurized liquid extraction. The extraction cell was prefilled with methanol to check for possible leakage before setting the oven to the desired temperature. Extraction with methanol was carried out for a period of 20 minutes and 20 to 25 ml of methanol was collected into a 25 ml volumetric flask. In between runs, the system was washed with methanol for 5 minutes.

### HPLC Conditions

For all experiments, a Shimadzu LC 10 series (Kyoto, Japan) equipped with a binary gradient pump, autosampler, column oven and diode array detector was used. The gradient elution consists of mobile phase of A)25 mM NaH₂PO₄ at pH 2.5 and B) acetonitrile. The initial condition was set at 30% of B, gradient up to 100% B in 15 minutes before returning to initial condition for 10 minutes. Detection was at 254 nm. Oven temperature was set at 40° C. and flow rate was set at 1.0 ml/min For all experiments, 20 μl of standards and sample extract were injected The column used for separation was Hypersil Elite (Runcorn, Cheshire, England) C18 (250×4.6 mm ID, 5 μl).

### EXAMPLE 2

### Comparison of Results

Pressurized Liquid Extraction of Baicalein in Scutellariae Radix:

From previous studies, the parameters that had a significant effect on the extraction efficiencies of marker compounds in botanicals and herbal preparations are the applied temperature and the solvent used The time for extraction was set at 20 minutes as it was found that a significant portion of the target analytes would be extracted in the first 20 minutes. The pressure was reported to have little effect on the extraction efficiency as it was applied to keep the solvent in the liquid phase. Stated differently, the main purpose of the applied pressure was to keep the solvent in the liquid state at a temperature above 100° C. [10, 19, 20, 21, 22]. The solvent selected was methanol as baicalein was reported to be soluble in methanol and practically insoluble in water [23].

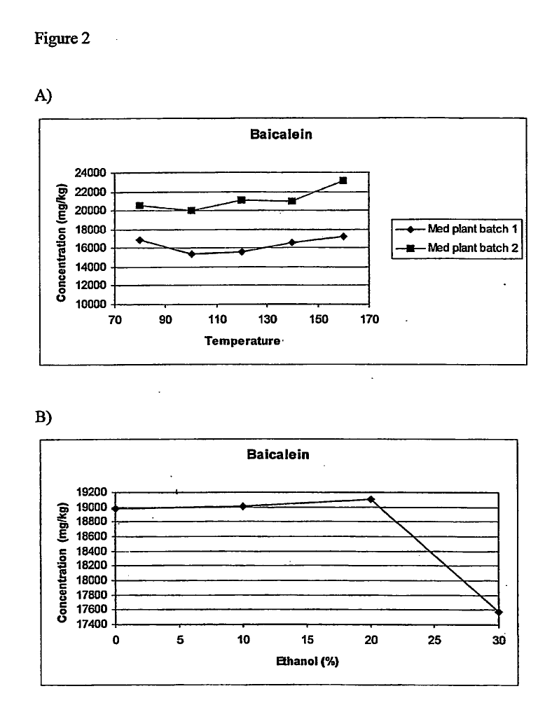

The effect of the applied temperature from 80 to 160° C. on the extraction efficiencies of baicalein from Scutellariae radix is shown in FIG. 2, part A. The data show that there is no significant increase in the extraction efficiencies in the range of temperature studied and baicalein was rather stable at temperatures up to 160° C. Hence, 140° C. was selected as the optimum temperature for the extraction of baicalein from Scutellariae radix.

The extraction efficiency by PLE was compared with that by soxhlet extraction. Previous work indicated that comparable or higher extraction efficiencies were obtained for berberine, aristolochic acids, strychnine, ginsenosides and glycyrrhizin in medicinal plants or herbal preparations with reference to sonication or soxhlet extraction [10, 19, 20, 21, 22]. The data in Table 4 shows that the extraction efficiency of PLE in 20 minutes was comparable to that obtained b soxhlet extraction for 3 to 4 hours. The method precision was found to vary from 2.4 to 4.6% (n=4/6) for different batches of medicinal plants on different days.

TABLE 1
Extraction of baicalein from medicinal plant (scutellariae
radix) by soxhlet extraction, PLE and PHWE.

| Mode of Extraction | Concentration of baicalein mg/g |
|---|---|
| Medicinal plant 1 by PLE | 20.21 ± 0.94 (RSD = 4.7%, n = 4) |
| Medicinal plant 1 by soxhlet | 25.82 ± 0.62 (n = 2) |
| extraction | |
| Medicinal plant 2 by PLE | 20.81 ± 0.49 (RSD = 2.4%, n = 4) |
| Medicinal plant 2 by soxhlet | 20.91 ± 0.39 (n = 2) |
| extraction | |
| Medicinal plant 3 by PLE | 18.27 ± 0.84 (RSD = 4.6%, n = 6) |
| Medicinal plant 3 by soxhlet | 18.70 ± 0.05 (n = 2) |
| extraction | |
| Medicinal plant 1 by PHWE | 24.28 ± 2.85 (RSD = 11.7%, n = 6) |
| Medicinal plant 1 by soxhlet | 24.44 ± 0.22 (n = 2) |
| extraction | |
| Medicinal plant 2 by PHWE | 22.81 ± 1.66 (RSD = 7.3%, n = 3) |
| Medicinal plant 2 by soxhlet | 25.45 ± 0.04 (n = 2) |
| extraction | |

Without being bound by theory, the comparable performance of PLE in comparison to soxhlet extraction is believed to be due to the higher solubility of analytes in solvent and higher diffusion rate as a result of higher temperature. At higher temperature, the strong solute—matrix interaction in the plant materials caused by van der Waals forces, hydrogen bonding and dipole attractions between solute molecules and active sites on the matrix are believed to be disrupted.

Pressurized Hot Water Extraction (PHWE) of Baicalein from Scutellariae Radix

The parameters that would have a significant effect on the extraction efficiencies of marker compounds in botanical materials using PHWE are the applied temperature, pressure and percentage of organic modifiers [11, 12, 13]. Plant materials were observed to have a strong tendency to adsorbed water during PHWE. The ground plant materials was thus dispersed evenly with sand to prevent any blockage of the extraction system. This step was not required for PLE.

From other references [11,13], water has similar properties as methanol at a certain pressures and temperatures between 150 to 200° C. But for PHWE of baicalein from scutellariae radix, a temperature of 95° C. was used.

Ethanol as organic modifier was added into the liquid water used in PHWE. The effects of ethanol (0 to 30%) are shown in FIG. 2, Part B. The data in FIG. 2B show that the extraction efficiencies of baicalein from scutellariae radix increased slightly with increasing percentage of ethanol added into the extraction water. Hence, 20% ethanol in water was selected for comparison with soxhlet extraction.

The extraction efficiencies of baicalein from scutellariae radix by PHWE were found to be comparable with that by soxhlet extraction as shown above in Table 1. The method precision (RSD) was found to vary between 7.3 to 11.7%. Higher method precision was observed for PHWE compared to PLE as a premixing step was required for sample preparation in PHWE. Although it was reported that baicalein was insoluble in water [23], the results herein show that water with a small percentage of ethanol at a temperature below its boiling point and a small applied pressure was able to extract an equivalent amount of baicalein in medicinal plants compared to soxhlet extraction with aqueous organic solvent. The results obtained by PHWE were in agreement with that using PLE with methanol as the extraction solvent.

Pressurized Hot Water Extraction of Glycyrrhizin from Radix Glycyrrhizae

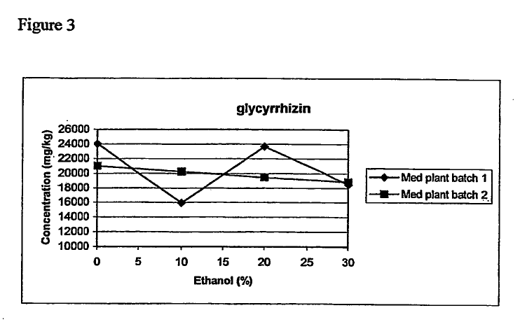

Because glycyrrhizin will degrade at temperatures above 120° C. using PLE with methanol as the solvent, a temperature of 100° C. was previously used for its extraction [10]. Glycyrrhizin was reported to be freely soluble in hot water and alcohol [23]. Hence, an applied temperature of 95° C. was selected for the initial experiments with PHWE. The effects of ethanol (0 to 30%) on the extraction efficiencies of PHWE was determined, and the results are shown in FIG. 3. The data show that the ethanol added did not increase the extraction efficiencies of glycyrrhizin in radix glycyrrhizae. Hence, PHWE using 100% water was selected for comparison with sonication.

The extraction efficiencies of PHWE were compared with sonication using methanol/water (70:30) and comparable or higher extraction efficiencies were observed for glycyrrhizin from radix glycyrrhizae as shown in Table 2.

TABLE 2
Extraction of Glycyrrhizin and berberine from
medicinal plant by PHWE and sonication.

| | Concentration of |
|---|---|
| Mode of extraction | target analytes, mg/g |
| Glycyrrbizin in radix glycyrrhizae 1 | 18.40 ± 0.64 (RSD = 3.5%, n = 5) |
| by PHWE | |
| Glycyrrhizin in radix glycyrrhizae 2 | 18.54 ± 0.48 (n = 2) |
| sonicaton | |
| Glycyrrhizin in radix glycyrrhizae 2 | 17.21 ± 1.27 (RSD = 7.4%, n = 5) |
| by PHWE | |
| Glycyrrhizin in radix glycyrrhizae 2 | 13.20 ± 2.37 (n = 2) |
| by sonication | |
| Berberine in coptidis rhizoma by | 62.72 ± 2.84 (RSD = 4.5%, n = 5) |
| PHWE at 95° C. | 60.69 ± 2.04 (RSD = 3.4%, n = 5) |
| Berberine in coptidis rhizoma | 80.72 ± 1.91 (n = 2) |
| by sonication | |
| Berberine in coptidis rhizoma by | 71.90 ± 1.68 (RSD = 2.3%, n = 5) |
| PHWE at 140° C. | |
| Berberine in coptidis rhizoma by | 74.13 ± 1.78 (RSD = 2.4%, n = 6) |
| PHWE at 140° C. with 20% ethanol | |

Similarly, higher extraction efficiencies compared to sonication were observed in previous experiments using PLE with methanol as the solvent [10]. The method precision (RSD) was found to vary between 3.5 to 7.4% for different batches of medicinal plants. PHWE thus provide an excellent means of extracting compounds such as glycyrrhizin that are soluble in hot water from medicinal plants.

Pressurized Hot Water Extraction (PHWE) of Berberine from Coptidis Rhizoma

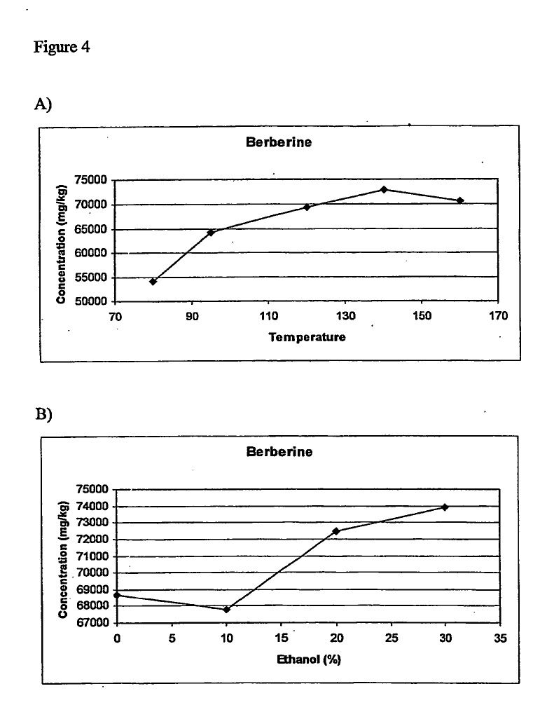

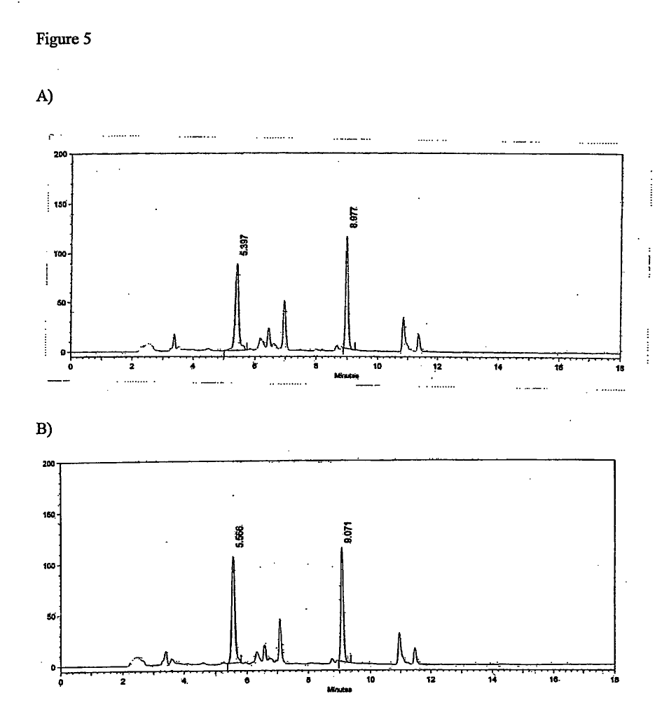

Berberine was reported to dissolve slowly in water [23]. The effects of the applied temperature from 80 to 160° C. on the extraction efficiencies of berberine in coptidis rhizoma are shown in FIG. 4, part X The results show that extraction efficiencies increased with the applied temperature from 80 to 140° C. Signs of minor degradation was observed at 160° C. as lower amounts of berberine was extracted compared to 140° C. Similarly, the effects of ethanol added on the extraction efficiencies at 95° C. was investigated and the results are shown in FIG. 4, part B. The results show that the extraction efficiencies were observed to increase with increasing amounts of ethanol in the water solvent.

The amount of berberine extracted from medicinal plant by PHWE using different conditions were compared with sonication and the results are tabulated in Table 2 above. At 95° C. and zero percent ethanol added, PHWE extracted approximately 75% of the berberine in comparison to sonication. At a higher temperature of 140° C., the amount of berberine extracted was approximately 90% compared to sonication. From Table 2, the addition of 20% ethanol in the liquid used did not increase the extraction efficiencies significantly. The method precision (RSD) was found to vary from 2.3 to 4.5%. These results show that the properties of water may be modified using a higher applied temperature with pressure to extract comparable amounts of berberine from medicinal plants.

Conclusions

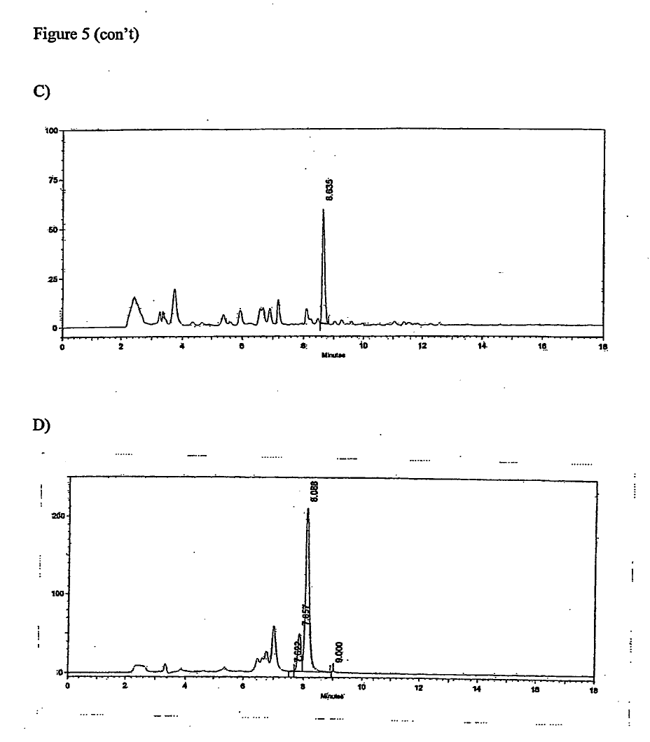

The above demonstrates that reasonably polar and thermal labile components in medicinal plants could be extracted using pressurized hot water at a temperature below 100° C. with pressure at levels comparable other reported methods [14, 15, 16, 17, 18]. Chromatograms of the various plant extracts from PHWE are shown in FIG. 5 and are seen to have a similar profile as extracts using organic solvents. The PHWE method used in the present work is a simpler system compared to previous reports, some of which required a second pump to pump organic solvent to flush through any organic compounds that precipitated when the plant extracts leave the oven and a cooling device was used to condense the plant extracts [12, 16, 17, 18]. The additional step of dispersing the plant samples with sand in PHWE contributed to good method precision and accuracy for the plant materials studied. PHWE may thus be applied as a method in the validation of botanicals, herbal preparations and dietary supplement, especially given the requirement for little or no organic solvent in the extraction process.

References

[1] United States Pharmacopeia & National Formulary, USP 24, NF 19, United States Pharmacopeial Convention, Inc, Rockville, 2000.

[2] Pharmacopoeia of the People's Republic of China, English Edition, The Pharmacopeia Commission of PRC, Beijing, 1997.

[3] WHO monographs on selected medicinal plants, Vol. 1, WHO Publications, Geneva, 1999.

[4] The Japanese Pharmacopeia, 13^{th} Edition, JP XIII, The society of Japanese Pharmacopeia, Japan, 1996.

[5] M. Blumenthal, A. Goldberg, J. Brinckmann, Herbal Medicine, expanded Commission E monographs, American Botanical Council, Austin, 2000.

[6] Guidance for Industry, Botanical Drug Products, draft guidance, CDER/USFDA, 2000.

[7] CPMP/CVMP, Note for guidance on specifications: Test procedures and acceptance criteria for herbal drugs, herbal drug preparations and herbal medicinal products, EMRA, 2000.

[8] F. Li, S. Sun, J. Wang, D. Wang, Biomedical Chromatography 12 (1998) 78-85.

[9] H. J. Issaq, Electrophoresis 20 (1999) 3190-3202.

[10] E. S. Ong, Electrophoresis, submitted

[11] S. B. Hawthorne, Y. Yang, D. J. Miller, Anal. Chem. 66 (1994) 2912-2920

[12] D. J. Miller, S. B. Hawthome, Anal. Chem. 70 (1998) 1618-1621

[13] B. van Bavel, K. Hartonen, C. Rappe, M. J. Riekkola, Analyst 124 (1999) 1351-1354

[14] V. Fernandez-Perez, M. M. Jimenez-Carmona, M. D. Luque de Castro, Analyst 125 (2000) 481-485

[15] M. M. Jimenez-Carmona, J. L. Ubera, M. D. Luque de Castro, J. Chromatogr. A 855 (1999) 625-632

[16] A. Basile, M. M. Jimenez-Carmona, A. A. Clifford, J. Agric. Food Chem. 46 (1998) 5205-5209

[17] J. Suomi, H. Siren, K. Hartonen, M. L. Riekkola, J. Chromatogr. A 868 (2000) 73-83

[18] A. Kubatova, D. J. Miller, S. B. Hawthorne, J. Chromatogr. A 923 (2001) 187-194

[19] E. S. Ong, S. O. Woo, Electrophoresis 22 (2001) 2236-2241

[20] E. S. Ong, S. N. binte Apandi Electrophoresis 22 (2001) 2723-2729

[21] E. S. Ong, S. O. Woo, Y. L. Yong, J. Chromatogr. A 904 (2000) 57-64

[22] H. K. Lee, H. L. Koh, E. S. Ong,. S. O. Woo, J. Sep. Sci. in press

[23] The Merck Index, 11 edition, Merck & Co., Inc, Rahway, N.J., 1989

[24] C. Y. Chu, S. J. Sheu, J. Chromatogr. A. 756 (1996) 137-144

[25] T. M. Pawlowski, Journal of Agricultural & Food Chemistry, (1998),46,3124-3132; D. J. Miller, S. B. Hawthorne, Journal of Chemical & Engineering Data, (2000) 45(2): 314318; R. S. Ayala, M. D. Luque de Castro, Food Chemistry, (2001) 75, 109-113; L. Gamiz-Gracia, M. D. Luque de Castro, Talanta (2000) 51, 1179-1185; Y. Yang, S. B. Hawthorne, D. J. Miller, Environmental Science & Technology (1997), 37, 430-4371; M. S. S. Curren, J. W. King, Journal of Agricultural & Food Chemistry, (2001), 49, 2175-2180; A. Di Corcia, A. B. Caracciolo, C. Crescenzi, G. Giuliano, S. Murtas, R. Samperi, Environmental Science & Technology, (1999), 33,3271-3277; C. Crescenzi, G. D'Ascenzo, A. Di Corcia, M. Nazzari, S. Marchese, R. Samperi, Analytical Chemistry, (1999), 71, 2157-2163; M. D. Luque de Castro, M. M. Jimenez-Carmona, V. Fernandez-Perez, Trends in Analytical Chemistry, (1999) 18(11): 708-716; V. Femandez-Perez, M. M. Jimenez-Carmrona, M. D. Luque de Castro, Atomic Spectrometry, (1999) 14, 1761-1765; J. A Field, K. Monohan, R. Reed, Analytical Chemistry, (1998), 70, 1956-1962; A. E. McGowin, K. K. Adom, A. K. Obubuafo, Chemosphere, (2001) 45, 857-864; B. Li, Y. Yang, Y. Gan, C. D. Eaton, P. He, A. D. Jones, Journal of Chromatography A, (2001) 873, 175-184; and U.S. Pat. No. 6,001,256.

All references cited herein are hereby incorporated by reference in their entireties, whether previously specifically incorporated or not. As used herein, the terms “a”, “an”, and “any” are each intended to include both the singular and plural forms.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

1. A method for water mediated extraction of analytes from a sample, said method comprising,
dynamically contacting an analyte containing sample with water below 100 degrees Celsius and at a regulated pressure from about 10 to about 30 bar.

2. The method of claim 1 wherein said water has a temperature above about 30 but below 100 degrees Celsius.

3. The method of claim 1 wherein said water contains an amount of at least one organic solvent.

4. The method of claim 1 wherein said regulated pressure is from about 10 to about 25 bar.

5. The method of claim 1 wherein said temperature is about 95 degrees Celsius.

6. The method of claim 1 wherein the pressure is selected from 10, 15, 20, or 25 bar.

7. The method of claim 1 wherein said sample comprises a dispersant

8. The method of claim 8 wherein said dispersant is selected from sand and glass beads.

9. The method of claim 1 wherein said contacting occurs for between about 5 and about 200 minutes.

10. The method of claim 1 wherein said contacting occurs for about 20 to about 40 minutes.

11. The method of claim 3 wherein said organic solvent is ethanol at a concentration of less than 50%.

12. The method of claim 11 wherein the concentration is selected from 5%, 10%,. 15%, 20%, 25%, and 30%.

13. The method of claim 1 wherein the sample is selected from a botanical or herbal preparation.

14. The method of claim 1 wherein said analytes include at least one analyte selected from berberine, baicalein and glycyrrhizin.

15. The method of claim 1 further comprising detection of said analytes.

16. The method of claim 1 wherein the dynamically contacting is at a flow rate of about 1 ml/min solvent.

17. A method of analyzing analytes extracted from a sample comprising the method of claim 1 further comprising analysis of said analytes by a technique selected from the group consisting of gas chromatography, mass spectrometry, ion chromatography, liquid chromatography and capillary electrophoresis.

18. The method of claim 1 wherein said water contains one or more surfactant or detergent.

19. The method of claim 1 wherein said water contains a detergent selected from sodium dodecyl sulfate and Triton X-100.

20. The method of claim 19 wherein said detergent is sodium dodecyl sulfate.
